(19) United States
(12) Patent Application Publication
(10) Pub. No.: US 2008/0109012 A1
(43) Pub. Date: May 8, 2008

(54) SYSTEM, METHOD AND APPARATUS FOR TABLESIDE REMOTE CONNECTIONS OF MEDICAL INSTRUMENTS AND SYSTEMS USING WIRELESS COMMUNICATIONS

(75) Inventors: Francois E. Falco, South Jordan, UT (US); Richard A. Leparmentier, Salt Lake City, UT (US); Vernon T. Jensen, Draper, UT (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/556,585

(22) Filed: Nov. 3, 2006

(51) Int. Cl. A61B 19/00

(52) U.S. Cl. 606/130

(57) ABSTRACT

System, method and apparatus are provided through which in some embodiments, a tool interface unit (TIU) of a medical navigation system or an integrated medical imaging and navigation system includes a wireless transceiver to communicate with a navigation computer in order to reduce or eliminate cabling between the TIU and the navigation computer. In some embodiments, the TIU is mounted to a side of a surgical table in order to reduce the possibility of contamination of the cables from a non-sterile environment. In some embodiments, the TIU is battery powered and includes a battery and a battery charger.

### BACKGROUND OF THE INVENTION

This disclosure relates generally to medical instruments and systems, and more particularly to a medical navigation system or an integrated medical imaging and navigation system having a tool interface unit (TIU) that is mounted proximate to a surgical table, provides wireless communication to a navigation computer and includes a cable management apparatus.

Medical navigation systems have enabled many types of medical operations to be performed using less invasive techniques. These systems are often used during image-guided surgery. Medical navigation systems track the precise location of surgical instruments in relation to multidimensional images of a patient's anatomy during medical procedures. Additionally, medical navigation systems use visualization tools to provide the surgeon with co-registered views of the surgical instruments in combination with the patient's anatomy. This functionality is typically provided by including components of the medical navigation system on a wheeled cart (or carts) that can be moved throughout the operating room.

The three most common position measurement technologies utilized in medical navigation systems are 1) mechanical, 2) optical, and 3) electromagnetic (EM). Regardless of the technology employed, most of these systems include a tool interface unit (TIU), which serves as an interface and distribution unit of the navigation system, and provides isolation between certain surgical instruments and the tracking electronics of the navigation system. Conventional TIUs typically include a plurality of inputs for receiving a plurality of input cables and a single output for coupling (connecting) to an output cable. The output cable is operably coupled to a navigation computer of the navigation system. On occasion, the cables that couple to the TIU are changed and/or reconfigured. Some surgical procedures require a variety of medical instruments and devices to be plugged and unplugged during the surgical procedures. However, conventional TIUs are typically located on the floor, under the surgical table. This location makes the TRI difficult to access when reconfiguring cables and adds to the clutter of the operating room by having a plurality of excess cable lengths lying on the floor. This exposes the TIU and its cables to a non-sterile environment that may include biological contaminants.

The number of cables, hoses and general clutter near the surgical table is a concern in an operating room. Cables on the floor near the surgical table, interfere with patient access by the surgical staff, as well as access to and positioning of mobile operating room equipment (e.g., imaging systems, monitoring systems, etc.). To ensure positioning flexibility, most cable lengths are designed for “worst case” position scenarios, which normally mean extra cable being coiled on the floor, adding to the clutter in the operating room.

For the reasons stated above, and for other reasons stated below which will become apparent to those skilled in the art upon reading and understanding the present specification, there is a need in the art for an easier method of connecting the TIU to the navigation computer and other devices of a medical navigation system or an integrated medical imaging and navigation system. There is also a need to reduce the number of cables that are connected to the TIU and shorten the cable lengths of the cables connected to the TIU. There is also a need to reduce exposure of the TIU and cables to a non-sterile environment and biological contaminants.

### BRIEF DESCRIPTION OF THE INVENTION

In one aspect, an apparatus for use in navigated surgical procedures, the apparatus comprising a tool interface unit (TIU) and a navigation computer operably coupled to the TIU via a wireless communication path.

In another aspect, a TIU comprising a processor and at least one wireless transceiver operably coupled to the processor, the at least one wireless transceiver operable to establish a wireless communication path to a navigation computer.

In yet another aspect, a medical navigation system comprising a TIU and a workstation operably coupled to the TIU via a wireless communication path.

In still another aspect, a method for navigated surgical procedures, the method comprising mounting a TIU to a side of a table, establishing a wireless communication path between the TIU and a navigation computer, and transmitting data between the TIU and the navigation computer via the wireless communication path.

Various other features, objects, and advantages of the invention will be made apparent to those skilled in the art from the accompanying drawings and detailed description thereof.

### BRIEF DESCRIPTION OF THE DRAWINGS

FIG. 1 is a simplified block diagram of a portion of a medical navigation system illustrating the interface between a tool interface unit (TIU) and a navigation computer according to an exemplary embodiment;

FIG. 2 is a simplified block diagram of a portion of a medical navigation system illustrating the interfaces between a TIU, a navigation computer, and a plurality of instruments and devices according to an exemplary embodiment;

FIG. 3 is a simplified block diagram of a portion of a medical navigation system, similar to FIG. 2, illustrating the interfaces between a TIU, a navigation computer, and a plurality of instruments and devices according to another exemplary embodiment;

FIG. 4 is a block diagram of a TIU according to an exemplary embodiment;

FIG. 5 is a block diagram of a TIU according to another exemplary embodiment;

FIG. 6 is a block diagram of a TIU according to yet another exemplary embodiment;

FIG. 7 is a block diagram of a TIU according to still another exemplary embodiment;

FIG. 8 is a diagram of a surgical table having a TIU and a cable management apparatus attached to a side of the table according to an exemplary embodiment; and

FIG. 9 is a diagram of a medical navigation system having a TIU mounted to a side of a table according to an exemplary embodiment.

### DETAILED DESCRIPTION OF THE INVENTION

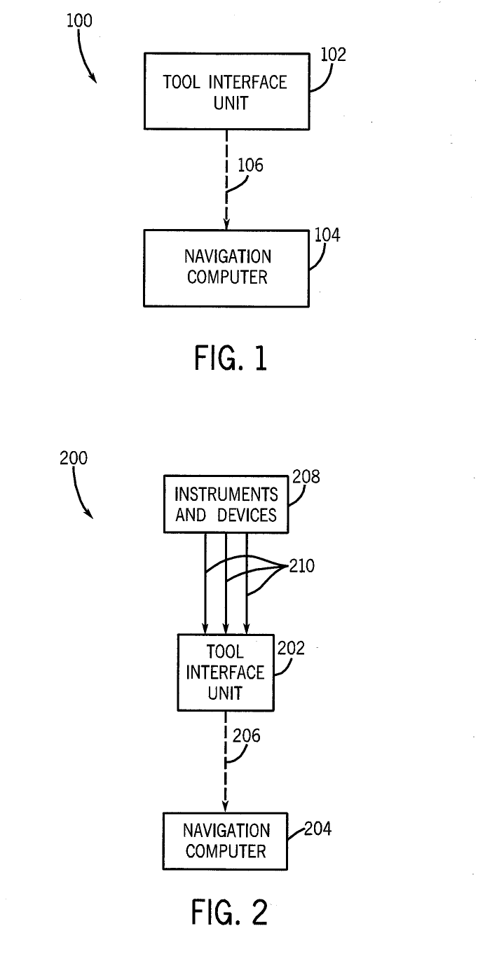

Referring to the drawings, FIG. 1 is a simplified block diagram of a portion of a medical navigation system 100 illustrating the interface between a tool interface unit (TIU) 102 and a navigation computer 104 according to an exemplary embodiment. Medical navigation system 100 solves the need in the art for connecting a TIU to a navigation computer and other devices using wireless communications.

Medical navigation system 100 includes a TIU 102 that serves as a cable interface and distribution unit for the navigation system. Medical navigation system 100 also includes a navigation computer 104 that is operably coupled via a wireless communication path 106 to the TIU 102. In some embodiments, the navigation computer 104 includes a navigation processor or a navigation central processing unit (CPU).

In some embodiments, the wireless communication path 106 replaces an entire output cable. In some embodiments, the wireless communication path 106 is a portion of the communication path 106 between the TIU 102 and the navigation computer 104, in which the wireless communication path 106 a replaces a potion of the output cable.

The wireless communication path 106 presents minimal physical impediments in connecting the TIU 102 to the navigation computer 104. Thus, the wireless communication path 106 between the TIU 102 and the navigation computer 104 solves the need in the art for a more convenient method of connecting the TIU 102 to a navigation computer.

While the system 100 is not limited to any particular TIU 102, navigation computer 104 or wireless communication path 106, for sake of clarity, a simplified TIU 102, navigation computer 104 and wireless communication path 106 are described.

FIG. 2 is a simplified block diagram of a portion of a medical navigation system 200 illustrating the interfaces between a TIU 202, a navigation computer 204, and a plurality of instruments and devices 208 according to an exemplary embodiment. The TIU 202 acts as an interface between a plurality of instruments and devices 208 and the navigation computer 204. The plurality of instruments and devices 208 may be medical instruments and devices, such as surgical instruments and navigation sensors used during navigated surgical procedures. The TIU 202 typically includes a plurality of inputs from a plurality of surgical instruments and/or a plurality of navigation devices, and a single output to the navigation computer 204. In this exemplary embodiment, the connections between the plurality of instruments and devices 208 and the TIU 202 are through a plurality of cables 210, and the connection between the TIU 202 and the navigation computer 204 is through a wireless communication path 206.

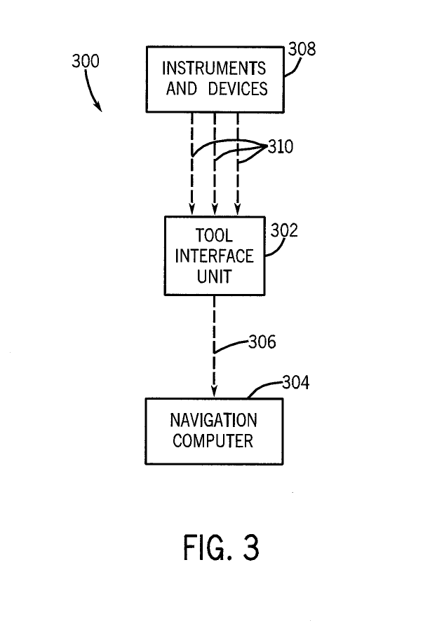

FIG. 3 is a simplified block diagram of a portion of a medical navigation system 300, similar to FIG. 2, illustrating the interfaces between a TIU 302, a navigation computer 304, and a plurality of instruments and devices 308 according to another exemplary embodiment. In this exemplary embodiment, the connections between the plurality of instruments and devices 308 and the TIU 302 are through wireless communication paths 310, and the connection between the TIU 302 and the navigation computer 304 is through a wireless communication path 306.

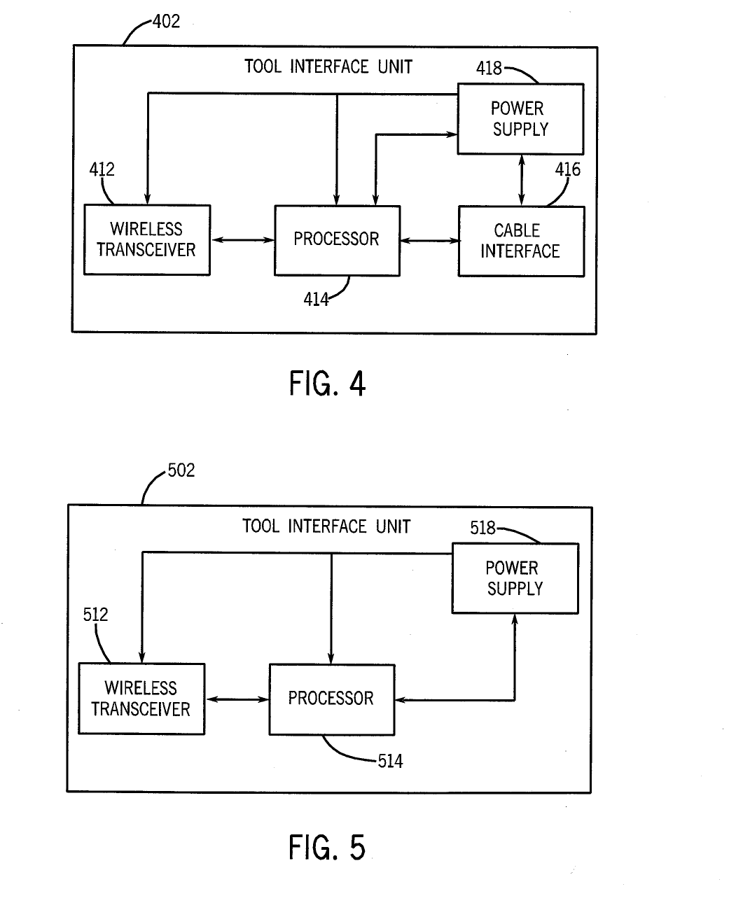

FIG. 4 is a block diagram of a TIU 402 according to an exemplary embodiment having a wireless transceiver 412. TIU 402 solves the need in the art to reduce the number of cables that are connected to the TIU by eliminating the cables connected between the TIU and the navigation workstation or computer.

TIU 402 includes a wireless transceiver 412 operably coupled to a processor 414. The wireless transceiver 412 is operable to establish a wireless communication path or link to a navigation computer, such as navigation computers 104, 204, 304 shown in FIGS. 1-3, and transmit and receive data between the TIU 402 and the navigation computer. For example, the wireless transceiver 412 may receive from processor 414, position and orientation information for each instrument and device coupled to the TIU 402, and transmits the information to the navigation computer. In some embodiments, the wireless transceiver 412 is a conventional wireless transceiver that communicates using over-the-air radio frequency signals.

The processor 414 is also operably coupled to a cable interface 416. The cable interface 416 is operable to pass data from a plurality of input cables operably coupled to a plurality of instruments and devices and processor 414. The plurality of input cables mechanically connect to cable interface 416. The cable interface 416 may include interface circuitry, distribution circuitry and isolation circuitry. The cable interface 416 is operable to isolate any of the plurality of instruments and devices that are in contact with the patient should an undesirable electrical short or surge take place.

In some embodiments, TIU 402 is powered by a power supply 418. The power supply 418 is operable to receive alternating current (AC) electrical power from an external power source (not shown), and supplies power to TIU 402 and all of its components, including wireless transceiver 412 and processor 414.

FIG. 5 is a block diagram of a TIU 502 according to another exemplary embodiment having at least one wireless transceiver 512. TIU 502 solves the need in the art to reduce the number of cables that are connected to the TIU by eliminating the cables connected between the instruments and devices and the TIU, and between the TIU and the navigation workstation or computer.

TIU 502 includes at least one wireless transceiver 512 operably coupled to a processor 514. The at least one wireless transceiver 512 is operable to establish a wireless communication path or link to a plurality of instruments and devices as inputs, and a navigation computer as an output. The at least one wireless transceiver 512 passes data between both the instruments and devices and the processor 514, and the processor 514 and the navigation computer. In some embodiments, the at least one wireless transceiver 512 is a conventional wireless transceiver that communicates using over-the-air radio frequency signals.

In some embodiments, TIU 502 is powered by a power supply 518. The power supply 518 is operable to receive AC electrical power from an external power source (not shown), and supplies power to TIU 502 and all of its components, including the at least one wireless transceiver 512 and processor 514.

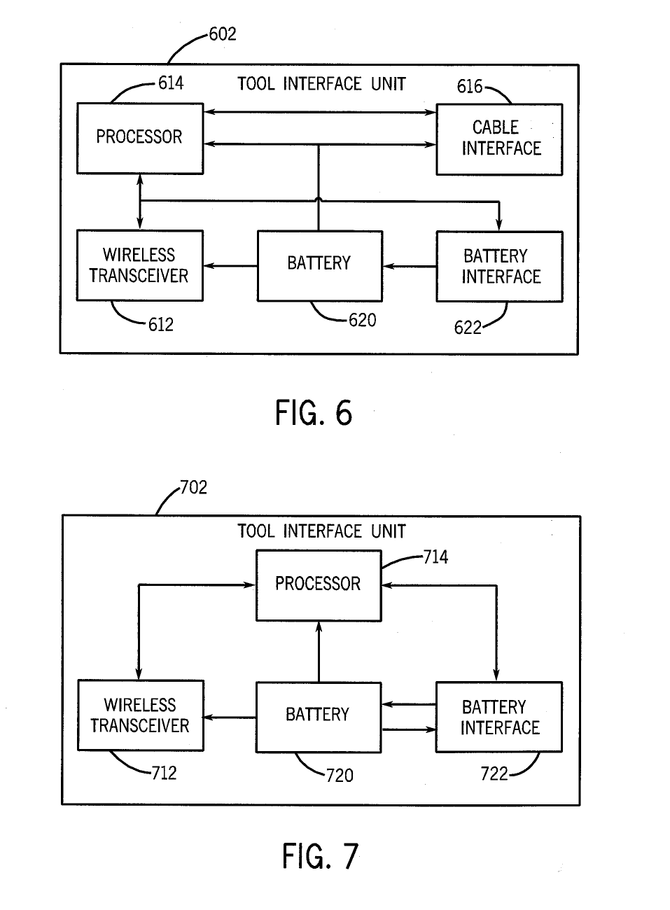

FIG. 6 is a block diagram of a TIU 602 according to yet another exemplary embodiment having a battery 620 for supplying power to the TIU, and a charging unit (battery interface 622) for recharging the battery. TIU 602 solves the need in the art to reduce the number of cables that are connected to the TIU.

TIU 602 includes a battery 620 that is operably coupled to a wireless transceiver 612, a cable interface 616 and a processor 614. The battery 620 is operable to supply electrical power to wireless transceiver 612, cable interface 616 and processor 614. In some embodiments, the battery 620 is a conventional off-the-shelf battery, and the battery is a rechargeable battery.

In some embodiments, the battery 620 is also operably coupled to a battery interface 622. The battery interface 622 is operable to receive AC electrical power from an external power source (not shown), and is operable to recharge battery 620. The battery interface 622 is also operably coupled to processor 614 for control and operation of the battery interface.

TIU 602 includes a wireless transceiver 612 operably coupled to the processor 614, operable to establishing a wireless communication path or link to a navigation computer. In some embodiments, the wireless transceiver 412 is a conventional wireless transceiver that communicates using over-the-air radio frequency signals. The processor 614 is also operably coupled to the cable interface 616. The cable interface 616 is operable to pass data from a plurality of instruments and devices to the processor 614.

FIG. 7 is a block diagram of a TIU 702 according to still another exemplary embodiment having a battery 720 for supplying power to the TIU, and a charging unit (battery interface 722) for recharging the battery. TIU 702 solves the need in the art to reduce the number of cables that are connected to the TIU. TIU 702 includes a battery 720 that is operably coupled to a wireless transceiver 712 and a processor 714. The battery 720 is operable to supply electrical power to wireless transceiver 712 and processor 714. In some embodiments, the battery 720 is a conventional off-the-shelf battery, and the battery is a rechargeable battery.

In some embodiments, the battery 720 is also operably coupled to a battery interface 722. The battery interface 722 is operable to receive AC electrical power from an external power source (not shown), and is operable to recharge battery 720. The battery interface 722 is also operably coupled to processor 714 for control and operation of the battery interface.

TIU 702 includes at least one wireless transceiver 712 operably coupled to a processor 714. The at least one wireless transceiver 712 is operable to establish a wireless communication path or link to a plurality of instruments and devices as inputs, and a navigation computer as an output. The at least one wireless transceiver 712 passes data between both the instruments and devices and the processor 514, and the processor 514 and the navigation computer. In some embodiments, the at least one wireless transceiver 712 is a conventional wireless transceiver that communicates using over-the-air radio frequency signals.

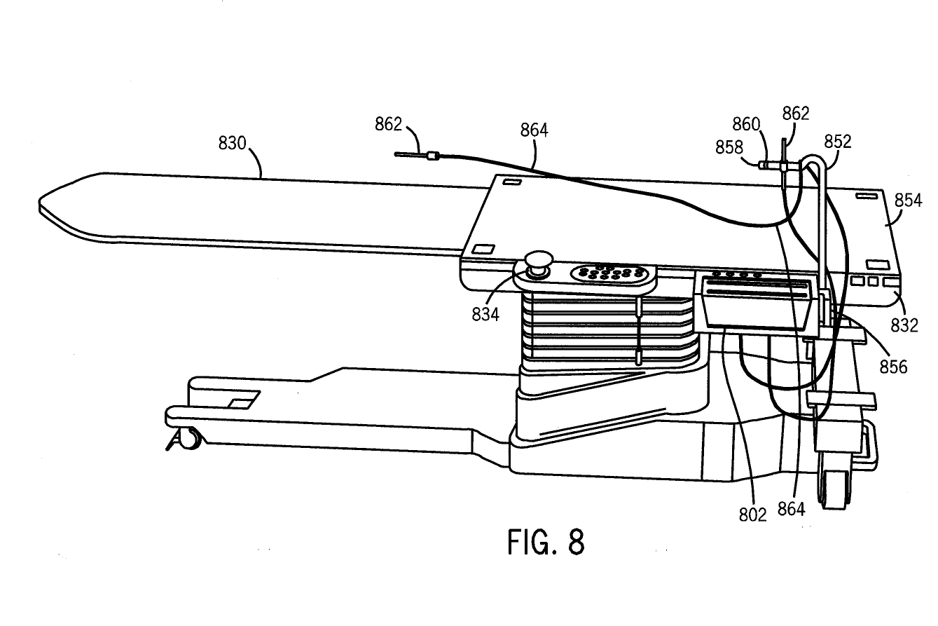

FIG. 8 is a diagram of a surgical table 830 having a TIU 802 and a cable management apparatus 852 attached to a side 832 of the table 830 according to an exemplary embodiment. TIU 802 and cable management apparatus 852 solve the need in the art to shorten the length of cables or eliminate cables interfacing with the TIU, and reduce exposure of the cables to a non-sterile environment that may include biological contaminants.

TIU 802 is mounted on or near the side 832 of the table 830, such as on a rail 834. The tableside mounted TIU 802 provides convenient access for connecting and disconnecting surgical instruments and navigation devices used during a navigated surgical procedure. The tableside mounted TIU 802 also keeps the TIU 802 and its connecting cables up off the floor in a sterile environment. This solves a common problem that occurs in many navigated surgical procedures, where plugging and unplugging of a variety of instruments and devices during the navigated surgical procedure is necessary. In some embodiments, the TIU may be mounted above or at an elevation higher than the table.

In some embodiments, a cable management apparatus 852 is attached to the side 832 of the table 830, adjacent to the TIU 802. The cable management apparatus 852 includes a shaft 854 extending upwardly from a base 856 and at least one arm 858 extending radially from one end of the shaft 854 opposite the base 856, for receiving the cable slack. The at least one arm 858 may include at least one holding portion 860 for holding an instrument or a device 862 that is not in use. The cable management apparatus 852 helps maintain a sterile field by managing slack in the cable 864 of a navigated instrument or device 862 that might otherwise be allowed to move in and out of the sterile field in a normal operating scenario. Cables 864 from the instruments or devices 862 that are connected to the TIU 802 may be wrapped around the at least one arm 858 of the cable management apparatus 852. The cable management apparatus 852 allows cable lengths to be shortened, further improving overall system quality, signal integrity and associated system accuracy. In some embodiments, the cable management apparatus 852 may include a flexible cable guide.

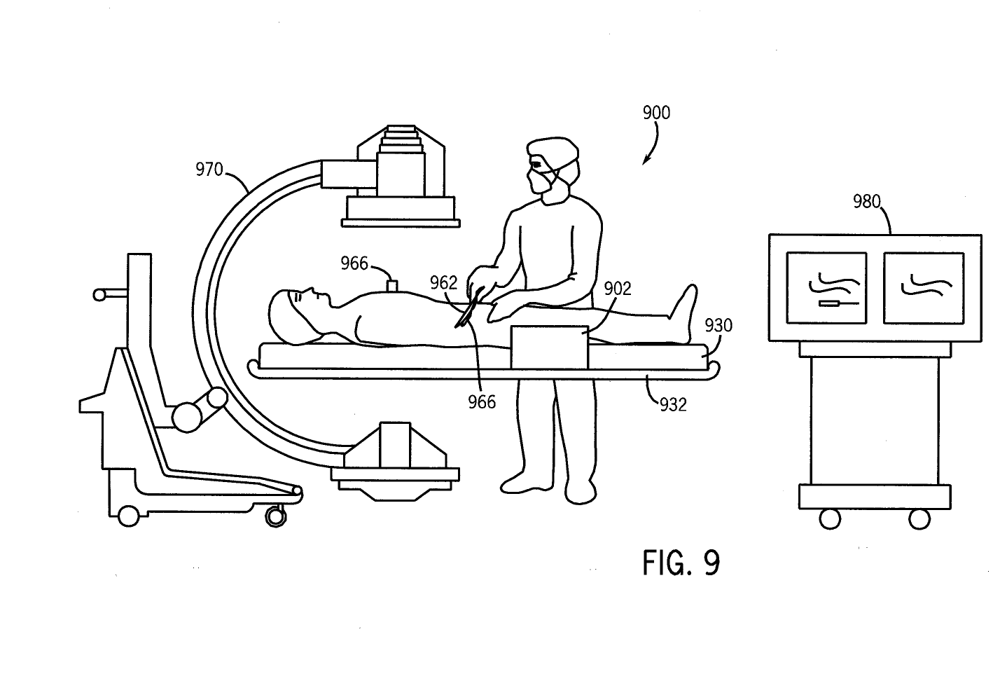

FIG. 9 is a diagram of a medical navigation system 900 having a TIU 902 mounted to a side 932 of a surgical table 930 according to an exemplary embodiment. Medical navigation system 900 solves the need in the art to shorten the length of cables or eliminate cables interfacing with the TIU, and reduce exposure of the cables to a non-sterile environment that may include biological contaminants. The medical navigation system 900 may be a stand-alone medical navigation system or an integrated medical imaging and navigation system.

The medical navigation system or integrated medical imaging and navigation system 900 includes an imaging system 970, a workstation 980, a TIU 902, at least one instrument 962, and/or at least one navigation sensor 966 positioned near a region of interest on a patient and/or mounted to at least one instrument 962. The workstation 980 may include imaging and navigation CPUs, navigation or tracking electronics, and at least one display. In some embodiments, communication between imaging system 970 and workstation 980 is through a wireless communication path; communication between at least one instrument 962 and at least one sensors 966, and TIU 902 is through a wireless communication path; and communication between TIU 902 and workstation 980 is through a wireless communication path.

The wireless communication technologies for connecting the different systems, instruments, and devices discussed herein may be, for example, Bluetooth, Wi-Fi, or any other wireless networking technology known in the art. The terminology used in this application is meant to include all communications environments and alternate technologies which provide the same functionality as described herein.

While the invention has been described with reference to a variety of embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

We claim:

1. An apparatus for use in navigated surgical procedures, the apparatus comprising:
a tool interface unit; and
a navigation computer operably coupled to the tool interface unit via a wireless communication path.

2. The apparatus of claim 1, further comprising a plurality of instruments and devices operably coupled to the tool interface unit.

3. The apparatus of claim 2, wherein the tool interface unit comprises at least one wireless transceiver and a processor operably coupled to the at least one wireless transceiver.

4. The apparatus of claim 3, wherein the tool interface unit further comprises a power supply operably coupled to the processor, the power supply operable to supply power to the processor and the at least one wireless transceiver.

5. The apparatus of claim 4, wherein the power supply is a battery.

6. The apparatus of claim 5, wherein the battery is a rechargeable battery.

7. The apparatus of claim 3, wherein the at least one wireless transceiver is operable to establish the wireless communication path between the tool interface unit and the navigation computer.

8. The apparatus of claim 3, wherein the at least one wireless transceiver is operable to establish a wireless communication path between the plurality of instruments and devices and the tool interface unit.

9. A tool interface unit comprising:
a processor; and
at least one wireless transceiver operably coupled to the processor, the at least one wireless transceiver operable to establish a wireless communication path to a navigation computer.

10. The tool interface unit of claim 9, further comprising a cable interface operably coupled to the processor, the cable interface operable to pass data between the processor and a plurality of cables, the plurality of cables being operably coupled to a plurality of instruments and devices.

11. The tool interface unit of claim 10, wherein the at least one wireless transceiver is operable to receive from the processor position and orientation information for each of the plurality of instruments and devices, and operable to transmit the position and orientation information to the navigation computer via a wireless communication path.

12. The tool interface unit of claim 9, wherein the at least one wireless transceiver is operable to establish a wireless communication path between a plurality of instruments and devices and the tool interface unit, and operable to transmit data between the plurality of instrument and devices and the processor.

13. The tool interface unit of claim 9, further comprising a power supply operably coupled to the processor, the power supply operable to supply power to the processor and the at least one wireless transceiver.

14. The tool interface unit of claim 13, wherein the power supply is a battery.

15. The tool interface unit of claim 14, wherein the battery is a rechargeable battery.

16. The tool interface unit of claim 15, further comprising a battery interface operably coupled to the battery, the battery interface operable to receive AC electrical power from an external power source, and operable to charge the rechargeable battery.

17. A medical navigation system comprising:
a tool interface unit; and
a workstation operably coupled to the tool interface unit via a wireless communication path.

18. The medical navigation system of claim 17, wherein the medical navigation system is an integrated medical imaging and navigation system.

19. The medical navigation system of claim 18, further comprising an imaging system operably coupled to the workstation.

20. The medical navigation system of claim 17, further comprising a cable management apparatus mounted adjacent to the tool interface unit for supporting excess cable lengths.

21. The medical navigation system of claim 17, wherein the tool interface unit is mounted to a side of a table.

22. The medical navigation system of claim 21, wherein the table includes a rail and the tool interface unit is mounted to the rail on the side of the table.

23. The medical navigation system of claim 17, wherein the workstation includes a navigation computer operably coupled to the tool interface unit via a wireless communication path.

24. The medical navigation system of claim 17, further comprising at least one instrument and/or at least one navigation sensor operably coupled to the tool interface unit via a wireless communication path.

25. The medical navigation system of claim 17, wherein the tool interface unit comprises at least one wireless transceiver, and a processor operably coupled to the at least one wireless transceiver.

26. A method for navigated surgical procedures, the method comprising:
mounting a tool interface unit to a side of a table;
establishing a wireless communication path between the tool interface unit and a navigation computer; and
transmitting data between the tool interface unit and the navigation computer via the wireless communication path.
